(19) United States
(12) Patent Application Publication
(10) Pub. No.: US 2026/0240935 A1
(43) Pub. Date: Aug. 20, 2026

(54) Pelleted Complete Mixed Feed (PCMF) For Improving Lamb Quality And Method Of Preparation Thereof

(71) Applicants: Baotou Beichen Feed and Technology Inc., Baotou (CN); Inner Mongolia Agricultural University, Hohhot (CN)

(72) Inventors: Yang JIA, Hohhot (CN); Xuezhen Liu, Baotou (CN); Yanfei Guo, Baotou (CN); Ting Duan, Baotou (CN)

(21) Appl. No.: 19/055,137

(22) Filed: Feb. 17, 2025

(51) Int. Cl. A61K 36/899; A23K 10/30; A23K 20/132; A23K 20/174; A23K 20/20; A23K 20/22; A23K 20/24; A23K 40/20; A23K 40/25; A61K 31/07; A61K 31/355; A61K 31/47; A61K 31/593; A61K 33/04; A61K 33/08; A61K 33/10; A61K 33/18; A61K 33/24; A61K 33/26; A61K 33/30; A61K 33/32; A61K 33/34; A61K 36/28; A61K 36/48; A61K 36/55

(52) U.S. Cl. CPC A61K 36/899; A23K 10/30; A23K 20/132; A23K 20/174; A23K 20/22; A23K 20/24; A23K 20/30; A23K 40/20; A23K 40/25; A61K 31/07; A61K 31/355; A61K 31/47; A61K 31/593; A61K 33/04; A61K 33/08; A61K 33/10; A61K 33/18; A61K 33/24; A61K 33/26; A61K 33/30; A61K 33/32; A61K 33/34; A61K 36/28; A61K 36/48; A61K 36/55

(57) ABSTRACT

This application relates to the technical field of feed preparation. Provided is a pelleted complete mixed feed (PCMF) for improving lamb quality. Each kilogram of the feed includes the following raw materials in parts by weight: 540-600 parts of corn, 85-115 parts of a bran, 160-220 parts of stir-fried soybean meal, 50-75 parts of a safflower seed, 12-18 parts of a flaxseed, 80-90 parts of a sunflower seed hull, and 35-50 parts of a premix. The PCMF is processed step by step. Feed materials rich in unsaturated fatty acid are selected, mixed well, pelleted, and pretreated. The premix is centrifuged, a resulting supernatant is mixed with the feed materials and dehydrated to make pellets. Undissolved premix components are dried and coated with pretreated pellet feed, so that easily denatured substances can be protected from rapid oxidation by air, delaying material degeneration and ensuring lamb quality improvement.

### TECHNICAL FIELD

The present disclosure relates to the technical field of feed preparation, in particular to a pelleted complete mixed feed (PCMF) for improving lamb quality and a preparation method thereof.

### BACKGROUND

China is a major livestock country, and the mutton sheep industry is a key sector within its livestock industry. Lamb is known for its nutritional properties, including high-protein, low-fat, and low-cholesterol. In recent years, with the increasing demand for lamb, sheep husbandry in China develops rapidly and plays an important role in the development of agricultural economy. According to data from the National Bureau of Statistics, in 2023, 338.64 million sheep were slaughtered nationwide, with an increase of 2.4 million (0.7%) sheep compared with that of the previous year; the lamb yield was 5.31 million tons, with an increase of 70,000 tons (1.3%). In terms of annual stock, the amount of slaughter, and lamb yield, China ranks first in the world. In recent years, with the continuous improvement of people's living standards and the enhancement of awareness of healthy diet, consumers' demand for high-quality healthy lamb products is increasing day by day. Nowadays, mutton sheep farming has generally shifted from grazing to house feeding or semi-house feeding pattern. While this significantly improves the breeding efficiency, it also leads to problems such as the decline of lamb flavor and quality and the aggravation of goaty flavor. The nutritional level of the diet is one of the key factors affecting the lamb flavor. Mutton sheep rations are directly related to lamb quality, as the types of nutrients that mutton sheep absorb from the feed determine the flavor compounds deposited in bodies thereof. The protein and energy levels of the diet and the pasture type will affect the lamb flavor to varying degrees. It has been shown that the lamb will produce undesirable odors when exclusively silage is fed, which may be attributable to indole or lipid oxidation. When grain-based diets are used, ruminal fermentation from grain is transformed to propionic acid fermentation, and propionic acid, as a major sugar-producing substance, affects body metabolism and flavor precursors, and thus lamb flavor. Therefore, regulating the nutritional level of the diet has become an important way to improve the quality of house-fed lamb. In the present disclosure, feed materials selected mainly include corn, bran, stir-fried soybean meal, safflower seed, flaxseed, sunflower seed hull, and premix. Corn, known as the “king of energy feed”, has the highest energy available in cereal seeds, providing a steady stream of energy for the daily activities, growth and fattening of sheep. In addition, the linoleic acid content of corn reaches 2%, which is the highest in all grain feeds. It has become a commonly used feed material due to the characteristics of excellent palatability, no limitation of use, and low price. Bran, as a by-product of wheat-processed flour, is widely used in livestock and poultry production due to its loose texture, large volume, excellent palatability, rich nutritional value, and massive dietary fiber and non-starch polysaccharides capable of regulating intestinal health. Soybean meal, a by-product of soybean (mainly soybean and black soybean) after pressing oil, is a high-quality protein feed. However, raw soybean meal contains some hazardous substances, such as antitrypsin, urease, hemagglutinin, saponin, and goitrogen. Among them, antitrypsin is the most harmful to livestock and poultry. However, after the raw soybean meal is stir-fried, anti-nutritional factors can be eliminated, and the protein structure is altered, meeting the protein needs of sheep growth and muscle development. In the rumen, rumen microorganisms can ferment and decompose the proteins in the soybean meal to produce amino acids and other nutrients that can be absorbed and utilized by sheep. Sunflower seed hull is a roughage, mainly composed of cellulose and lignin (accounting for 50% of the total hull weight), reducing sugar (accounting for 25.7% of the total hull weight, which is the second largest component), and lipids (accounting for 5.17% of the total hull weight), 2.96% of which are vegetable waxes composed of long-chain fatty acids (C14-28, predominantly C20) and fatty alcohols (C12-30, predominantly C22, C24, and C26). Adding sunflower seed hulls to mutton sheep feed can improve rumen health and promote the growth of mutton sheep. It has been shown that addition of a specific level of fat to the diet not only increases the energy supply of ruminants, but also affects fat deposition and carcass composition. Additionally, it can be used to regulate muscle fatty acid composition by providing polyunsaturated fatty acids (PUFAs), thereby increasing the deposition and conversion efficiency of beneficial fatty acids in the product. Therefore, supplementing fat in the diet not only increases the energy concentration of ruminant feed, but also significantly improves meat quality. PUFAs in the diet can change the composition of rumen microorganisms, which in turn affect the composition of rumen fatty acid hydrogenation intermediates and rumen fatty acids, ultimately leading to changes in the composition of fatty acids in body tissues. However, dietary lipids that enter the rumen are rapidly hydrolyzed to release free fatty acids in the presence of microbial lipolytic enzymes. Unsaturated fatty acids are quickly hydrogenated into saturated fatty acids by microorganisms, which increases saturated fatty acids absorbed into the distal gut and bloodstream of ruminants, while reducing unsaturated fatty acids. Therefore, the corresponding fatty acids absorbed by the tissue from the blood will also change accordingly, ultimately reducing the quality of fatty acids in animal products. It is evident that reducing the hydrogenation of unsaturated fatty acids of ruminant diets in the rumen has become one of the important ways to improve the fatty acid composition of ruminant products. Therefore, in the present disclosure, safflower seeds and flaxseeds rich in unsaturated fatty acids are selected as feed materials. In the safflower seed, the oleic acid content is up to 13-21%, and the linoleic acid content is up to 73-79%. Linoleic acid is an essential unsaturated fatty acid that cannot be synthesized by the body. In addition, safflower seeds are rich in vitamin E, which can improve the vascular environment and has strong antioxidant effects; flaxseed is rich in unsaturated fatty acids, especially linolenic acid, which has a positive effect on the health and meat quality of sheep and increases the content of unsaturated fatty acids in lamb, thereby improving lamb quality. However, these raw materials, despite their high nutritional value, share a common “weakness”—they are prone to oxidation. Due to the rich content of unsaturated fatty acids in the raw materials, oxidation reactions will occur after prolonged exposure to air. If conventional production processes are used for processing, beneficial substances in the feed are highly susceptible to denaturation, reducing its palatability and affecting the normal feeding of sheep. This not only fails to improve the lamb quality, but also substantially reduces the utilization rate of high-quality feed resources, resulting in the waste of resources. In order to solve this problem, the present application adopts the process of centrifugation and coating. The premix is mixed with water and centrifuged for solid-liquid separation. Feed materials are soaked with the aqueous solution of minerals, dried to a semi-dry state, and initially coated. Then, the water-insoluble premix components are dried and coated with the outer layer of the semi-dry feed pellets to form a protective film. A two-stage drying method is used to form a dense layer on the surface of the feed pellets to reduce the contact between the easily deformed internal substance and the air, improving the antioxidant function of the product, and ensuring that the feed can maintain its nutritional value during storage and use. This provides sheep with stable and high-quality nutrition supply, thereby improving the lamb quality.

### SUMMARY

An objective of the embodiments of the present disclosure is to provide a PCMF for improving lamb quality and a method of preparation thereof, so as to solve the problem in the background that added nutrients cannot be stored for a long time to maintain nondegeneration.

To achieve the above objective, the present disclosure provides the following technical solutions. A PCMF for improving lamb is provided, where each kilogram of the feed includes the following raw materials in parts by weight: 540-600 parts of corn, 85-115 parts of a bran, 160-220 parts of a stir-fried soybean meal, 50-75 parts of a safflower seed, 12-18 parts of a flaxseed, 80-90 parts of a sunflower seed hull, and 35-50 parts of a premix.

Each part by weight of the premix includes the following components: 0.04-0.06% of copper sulfate pentahydrate, 0.18-0.23% of ferrous sulfate monohydrate, 0.24-0.32% of zinc sulfate monohydrate, 0.12-0.18% of manganese(II) sulfate monohydrate, 0.02-0.03% of calcium iodate monohydrate, 0.01-0.02% of sodium selenite, 0.01-0.02% of cobalt chloride monohydrate, 3-5% of magnesium oxide, 28-32% of calcium carbonate, 0.03-0.04% of retinyl acetate, 0.004-0.006% of vitamin D3, 0.50-1.0% of vitamin E, 0.25-0.35% of ethoxyquin, and 32-40% of a carrier.

Further provided is a method for preparing the PCMF for improving lamb quality, including the following steps:
step S1, pretreatment of the feed raw materials: putting the corn, the stir-fried soybean meal, the bran, the safflower seed, the flaxseed and the sunflower seed hull in the parts by weight into a grinder for grinding;
step S2, pretreatment of the premix components: feeding and mixing the copper sulfate pentahydrate, the ferrous sulfate monohydrate, the zinc sulfate monohydrate, the manganese (II) sulfate monohydrate, the calcium iodate monohydrate, the sodium selenite, the cobalt chloride monohydrate, the magnesium oxide, the calcium carbonate, the retinyl acetate, the vitamin D3, the vitamin E, the ethoxyquin, and the carrier in parts by weight and in batches into a mixer proportionally in a layered feeding manner, while slowly adding purified water equal to twice a total volume of additives to obtain a first wet mix;
step S3, centrifugation: feeding the first wet mix into a centrifuge, centrifuging for solid-liquid separation to obtain a first dry mix and a supernatant, and loading the first dry mix on a tray for later use;
step S4, mixing the supernatant with the feed raw materials obtained from step S1, and putting a resulting mixture in a grinder for grinding to obtain a second wet mix;
step S5, heating and drying the second wet mix until the second wet mix becomes viscous to obtain a second semi-dry mix;
step S6, extruding the viscous second semi-dry mix on a feed pellet pressing mill to obtain a second semi-dry mix pellet, and loading the second semi-dry mix pellet on a tray with the first dry mix; and
step S7, adhering the first dry mix to a surface of the second semi-dry mix pellet, and conducting drying to obtain a finished pellet feed.

In some embodiments, the feed raw materials in step S1 have a particle size smaller than 3 mm, each feed raw material is individually crushed, and feed raw materials are all mixed well in the mixer after crushing.

The above technical solution is adopted to ensure that the feed raw materials can be evenly distributed in each grain of feed.

In some embodiments, the premix components in step S2 have a particle size smaller than 1 mm, and the components are all mixed well in the mixer according to the above technical solution, enabling spontaneous and homogeneous distribution of the components during the grinding process.

In some embodiments, the centrifuge in step S3 is set at a rotational speed of 2,000-3,000 rpm, and centrifugation time is controlled in a range of 10-15 min.

The above technical solution is adopted to avoid serious dehydration of the components caused by excessively high centrifugal speed.

In some embodiments, the second wet mix in step S4 is wrapped with a cling film.

The above technical solution is adopted to ensure that materials will not be naturally air-dried when not in use.

In some embodiments, the drying in step S5 is set at 75-85° C., and a moisture of the second semi-dry mix is controlled in a range of 15-20%.

The above technical solution is adopted to ensure that the second semi-dry mix can be agglomerated and pelleted without natural dispersion.

In some embodiments, a particle size for the feed pellet pressing mill in step S6 is set at 3-8 mm.

Using the above technical solution, feed raw materials of different sizes are used to adapt to sheep at different growth stages for consumption.

In some embodiments, the drying in step S7 is divided into two stages of 200° C. and 80° C., and materials need to be continuously turned over during the drying.

Using the above technical solution, a dense layer is formed on the surface of feed pellet through two-stage drying to reduce the contact between easily deformed internal substances and air.

Compared with the prior art, embodiments of the present disclosure have the following beneficial effects: the PCMF for improving lamb quality has the following characteristics.

1. Using a step-by-step processing pattern, easily denatured feed raw materials are selected, mixed well, pelleted and pretreated, and the premix is dehydrated, dried and coated with pretreated pellet feed. Thus, easily denatured substances can be protected from being quickly oxidized by air, delaying material degeneration and ensuring the improvement of lamb quality.

Further, in the process of feed forming, the outer layer is dried and hardened rapidly at high temperature, and the inner layer is dried slowly by heat, so that the easily denatured substances in the inner layer of feed can be further blocked and the volatilization of easily denatured substances can be reduced.

Furthermore, adding safflower seeds can improve the muscle quality. The content of linolenic acid and n-3 long chain PUFAs in adipose tissue increases when flaxseeds are added to the diet for lambs during fattening, which is beneficial to the improvement of the lamb quality. With the increase of flaxseed addition, the meat tenderness also significantly increases. Dietary addition of magnesium oxide can reduce the fat content in the longissimus dorsi of the sheep and increase the content of myoglobin in the muscle, thereby improving the meat color of the lamb. Vitamin E is an important fat-soluble antioxidant capable of scavenging free radicals and preventing the oxidation reaction caused by non-free radical oxidants. The use of vitamin E as an antioxidant in the diet not only improves the antioxidant activity of feed raw materials, but also improves the meat quality by preventing the oxidation of PUFAs after fed to animals. Ethoxyquin has strong antioxidant activity and is one of the feed antioxidants with excellent performance. It can prevent the oxidative deterioration of oil and vitamins in feed, and is beneficial to the improvement of the color of animal products.

2. In the centrifugation process, the solid and liquid components can be effectively separated from the first wet mix and then recombined, so that the nutritional additives in the feed can be distributed in the feed pellets more accurately, while the first dry mix obtained by centrifugation and drying has a suitable pellet state, which is crucial for the subsequent pelleting process. The appropriate dry mix state can make the second wet mix easier to form during extrusion and pelleting, and the structure of the pellet is more compact and uniform.

Further, the excess liquid is separated by centrifugation, which reduces the water content in the feed and the possibility of microbial growth. The growth and propagation of microorganisms usually require certain humidity conditions. The humidity of the dry mix is low after centrifugation and drying. After the process of binding to the second semi-dry mix pellets under reasonable humidity control, the whole feed could maintain a low moisture.

Furthermore, the centrifugation will facilitate subsequent steps, such as grinding, mixing and drying. Because the first dry mix and supernatant resulted from centrifugation are more suitable for subsequent processing, the dry mix can reach a uniform state faster when mixed with other materials, while the supernatant can also be mixed well more smoothly after bound to the crushed feed raw materials, which reduces the processing difficulty due to the poor material state and improves the efficiency of the whole processing technology.

### BRIEF DESCRIPTION OF THE DRAWINGS

FIG. 1 is a flowchart of feed preparation in the present disclosure.

### DETAILED DESCRIPTION OF THE EMBODIMENTS

The embodiments of the present disclosure will be clearly and completely described below with reference to the drawings of the examples of the present disclosure. Apparently, the described examples are merely a part of, not all of, the embodiments of the present disclosure. All other embodiments obtained by a person of ordinary skill in the art based on the examples of the present disclosure without creative efforts shall fall within the protection scope of the present disclosure.

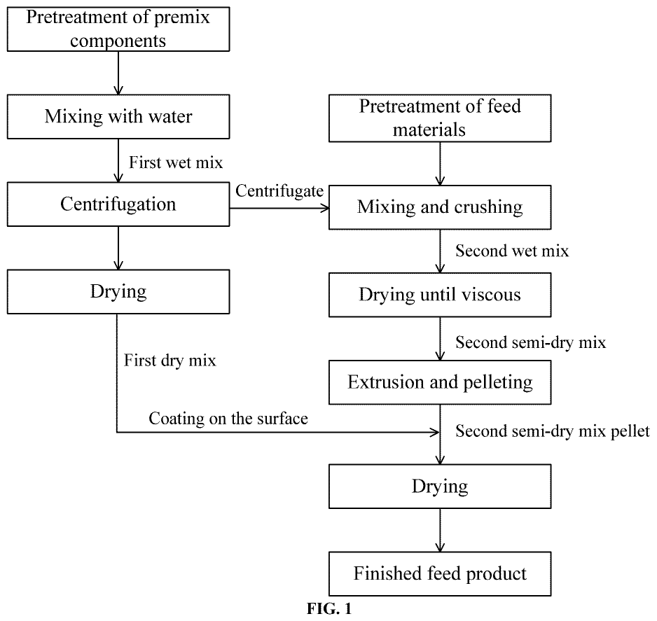

As shown in FIG. 1, the present disclosure provides an embodiment of the PCMF for improving lamb quality.

### Example 1

This example disclosed a PCMF for improving lamb quality. Each kilogram of the feed included the following raw materials in parts by weight: 552 parts of corn, 89 parts of bran, 168 parts of stir-fried soybean meal, 52 parts of safflower seed, 13 parts of flaxseed, 89 parts of sunflower seed hull, and 37 parts of additives.

### Example 2

This example disclosed a PCMF for improving lamb quality. Each kilogram of the feed included the following raw materials in parts by weight: 501 parts of corn, 98 parts of bran, 188 parts of stir-fried soybean meal, 64 parts of safflower seed, 15 parts of flaxseed, 85 parts of sunflower seed hull, and 49 parts of additives.

### Example 3

This example disclosed a PCMF for improving lamb quality. Each kilogram of the feed included the following raw materials in parts by weight: 526 parts of corn, 94 parts of bran, 179 parts of stir-fried soybean meal, 56 parts of safflower seed, 14 parts of flaxseed, 86 parts of sunflower seed hull, and 35 parts of additives.

Verification test of feed for improving lamb quality:

### 1. Changes in Body Weight of Mutton Sheep

This experiment began on Sep. 15, 2023. The experiment lasted for 90 days. A total of 120 healthy sheep with an average weight of about 24 kg were selected and divided into 3 groups (Example 1, 2, and 3 groups) of 40 sheep. During the experiment, the automatic feeding trough was used for feeding. The feed was supplemented every 3 days. Mutton sheep had ad libitum access to feed and water, and feed intake was recorded. At the beginning and end of the experiment, the sheep weights were measured and recorded. The experimental results are shown in Table 1.

TABLE 1
Changes in body weight of sheep

| Item | Example 1 | Example 2 | Example 3 |
|---|---|---|---|
| Average initial | 24.44 | 24.52 | 24.26 |
| weight, kg | | | |
| Average 90-day | 51.05 | 47.82 | 48.78 |
| body weight, kg | | | |
| Average daily gain, kg | 0.29 | 0.25 | 0.27 |

According to the data in Table 1, the average daily gain of sheep is ranked in descending order: Example 1 group>Example 3 group>Example 2 group.

### 2. Lamp Quality

Meat color: The meat color was measured using a colorimeter, indicators including the luminance (L*), redness (a*) and yellowness (b*).

Drip loss: Three mutton sheep were selected from each group. A piece of longissimus dorsi of about 5×3×2 cm was sampled from each sheep and weighed, and the weight was designated as the initial weight; then the meat sample was sealed in a plastic bag and hung in a refrigerator at 0-4° C. for 24 h. After the meat sample was taken out, the plastic bag was removed. The sample was weighed, and the weight was designated as the final weight.

$$Drip loss of lamb = ( Initial weight - Final weight ) / Initial weight \times 100 \%$$

Drip loss rate: Three sheep were selected from each group, and a 1 cm piece of thick longissimus dorsi was sampled from each sheep. Three round meat samples sampled using a sampler with a diameter of 2.532 cm were weighed. After covered with gauze, the samples were placed on 18 layers of qualitative medium-speed filter paper and plastic pads. The samples were pressurized to 35 kg with a pressure gauge. After 5 min, the samples were taken out, the water on the surface of the sample was wiped, and the samples were weighed immediately. The water content of the meat sample was obtained according to the direct drying method.

$$Drip loss rate = ( Meat sample weight before pressurization - Meat sample weight after pressurization ) / Meat sample weight before pressurization \times 100 \%$$

Cooking loss: Three sheep were selected from each group. Three longissimus dorsi samples each weighing 300-500 g were weighed, respectively. Each sample was put in a ziplock bag, exhausted (until no bubbles) in a water bath at 75° C. for 30 min (completely immersed), and cooled in running water at 15° C. for 40 min. The ziplock bag was opened, and the water on the surface of the meat sample was wiped before sample weighing

$$Cooking loss = ( Weight before water bath - Weight after water bath ) / Weight before water bath \times 100 \%$$

The measurement results detected by the above method are shown in Table 2.

TABLE 2
lamb quality

| Measurement | | | |
|---|---|---|---|
| method | Example 1 | Example 2 | Example 3 |
| Meat color L* | 35.81 | 32.05 | 36.21 |
| Meat color a* | 11.56 | 15.67 | 12.56 |
| Meat color b* | 7.01 | 5.35 | 6.28 |
| Drip loss % | 1.43 | 1.54 | 1.22 |
| Drip loss rate % | 21.26 | 22.51 | 20.03 |
| Cooking loss % | 28.92 | 30.04 | 26.55 |

Within a specific range of values, the smaller the values of L* and b* are, the greater the value of a* is, and the better the meat color is. As can be seen from the data in Table 2, meat color L*, meat color a*, meat color b*, drip loss, drip loss rate, and cooking loss in sheep muscle are ranked in descending order: Example 2 Group > Example 1 Group > Example 3 Group.

### 3. Fatty Acid Composition of Lamb

Three sheep were selected from each group, and 10 g of longissimus dorsi from each sheep was sampled and dried with a self-made sampler; 0.5 g of dried and crushed meat sample was add with 4 mL of isooctane, mixed well and put into a 37° C. constant temperature incubator shaker to shake overnight. Next, the meat sample was mixed with 4 mL of 2 mol/L potassium hydroxide in methanol for methyl esterification. Then, potassium hydroxide was neutralized with about 1 g of sodium bisulfate, followed by filtering through a 0.45 μm millipore filter and collecting the supernatant, which was then detected by high-performance gas chromatography. The chromatographic conditions were as follows. The chromatographic column was DB-WAX, 30 m×0.25 mm×0.5 m; split/splitless inlet (SPL) temperature was 250° C.; the injection method was split injection, the split ratio was 20:1, and the injection volume was 1 μL; the detector was a hydrogen flame ionization detector (FID), the temperature was 280° C., the carrier gas was nitrogen (N₂), the rate was 36 cm/s, the purge flow was 3 mL/min, and the makeup flow was 30 mL/min; the hydrogen flow was 40 mL/min, and the air flow was 400 mL/min; the column temperature was programmed as follows: 50° C. for 1 min, 25° C./min to 200° C., and 3° C./min to 230° C. for 16 min. The results of lamb fat content determined by the above method are shown in Table 3.

TABLE 3
Changes of muscle fatty acid composition

| Item | Example 1 | Example 2 | Example 3 |
|---|---|---|---|
| C18:0 (Stearic acid) | 17.72 | 14.59 | 16.53 |
| C18:1n9 (Oleic acid) | 35.46 | 42.51 | 34.08 |
| C18:1n7 (Oleic acid) | 1.62 | 1.88 | 1.54 |
| C18:3n6 (Linoleic acid) | 0.15 | 0.18 | 0.11 |
| C18:3n3 (Linoleic acid) | 0.37 | 0.52 | 0.35 |
| CLA-c9t11 (Conjugated linoleic | 0.30 | 0.48 | 0.42 |
| acid) | | | |
| CLA-c9t11 (Conjugated linoleic | 0.06 | 0.08 | 0.05 |
| acid) | | | |
| Short-chain fatty acids | 0.15 | 0.10 | 0.16 |
| Saturated fatty acids | 43.28 | 36.21 | 44.56 |
| Monounsaturated fatty acids | 39.16 | 45.27 | 36.05 |
| PUFAs | 15.07 | 18.55 | 13.93 |

Fatty acids of lamb are another determinant affecting meat quality. It is important to investigate the fatty acid composition of lamb to improve meat quality and flavor. According to the data in Table 3, the sheep fed the ration of Example 2 had higher content of C18: 1n9 (oleic acid), C18: 1n7 (oleic acid), C18: 3n6 (linolenic acid), C18: 3n3 (linolenic acid), CLA-c9t11 (conjugated linoleic acid), CLA-c9t11 (conjugated linoleic acid) monounsaturated fatty acids, and PUFAs in the muscle compared with other groups.

### 4. Detection of Nutrients in Lamb

For method for determination of cholesterol, national standard GB/T 5009.128-2016 was followed.

For method for determination of calcium, national standard GB/T 5009.92-2016 was followed.

### Method for Determination of Crude Protein:

Three sheep were selected from each group, and 20 g each of meat samples of different parts was placed in a Waring blender, crushed with 100 mL of 0.15 mol/L NaCl, put in a centrifuge, and centrifuged at 4000 r/min for 40 min. The supernatant (1 mL) was collected and diluted 200-fold; then 0.5 mL of diluent was sampled and 0.5 mL of 0.15 mol/L NaCl and 10 mL of Coomassie brilliant blue solution were added. After complete reaction, a sample was taken to measure its absorbance at 595 nm using Model 722 spectrophotometer.

Preparation of Coomassie brilliant blue: Accurately, 10 mg of Coomassie brilliant blue G250 was weighed and 5 mL of 95% ethanol solution was added until the solution became blue. Then 10 mL of 85% phosphoric acid solution was added to make the solution blood red. After the step of letting stand at room temperature, the solution was diluted with distilled water to 100 mL until it became brown. The solution was filtered with filter paper and stored in the dark for later use.

Plotting of standard curve: Separately, 0.0, 0.1, 0.2, 0.3, 0.5, 0.6, 0.7, and 0.9 mL of 0.1 mg/mL standard bovine serum albumin (BSA) solutions were made up to 1.0 mL with 0.15 mol/L NaCl solution, and reacted with 10.0 mL of Coomassie brilliant blue solution for a moment at room temperature. On a Model 722 spectrophotometer, the wavelength was adjusted to 595 nm, the reaction mixtures were placed in quartz cuvettes, and colorimetry was conducted with double distilled water as blank control. The formula obtained from the curve was Y=0.3411X, R²=0.996 (where X represents protein content and Y represents absorbance). 0.5 mL of 0.15 mol/L NaCl solution was added to the test tube, and then 10 mL of Coomassie brilliant blue solution was added. The absorbance was measured at 595 nm using Model 722 spectrophotometer.

$$X = Y / 0.3411 * 20 / 0.15 ( where X represents percentage content of protein , and Y represents absorbance )$$

The measurement results by the above method are shown in Table 4.

TABLE 4
Measurement results of nutrients in lamb

| Item | Example 1 | Example 2 | Example 3 |
|---|---|---|---|
| Protein % | 18.20 | 20.30 | 19.12 |
| Cholesterol, | 36.1 | 34.24 | 35.38 |
| mg/100 g | | | |
| Calcium, mg/kg | 82.55 | 85.32 | 84.56 |

According to the data in Table 4, it can be seen that the content of protein in lamb is ranked in descending order: Example 2>Example 3>Example 1.

The content of cholesterol in lamb is ranked in descending order: Example 1>Example 2>Example 1.

The content of calcium in lamb is ranked in descending order: Example 2>Example 3>Example 1.

The method for preparing a PCMF for improving lamb quality included the following steps:
step 1, pretreatment of the feed raw materials: the corn, the stir-fried soybean meal, the bran, the safflower seed, the flaxseed and the sunflower seed hull, in the parts by weight, were put into a grinder for grinding;
step 2, pretreatment of the premix components: the copper sulfate pentahydrate, the ferrous sulfate monohydrate, the zinc sulfate monohydrate, the manganese(II) sulfate monohydrate, the calcium iodate monohydrate, the sodium selenite, the cobalt chloride monohydrate, the magnesium oxide, the calcium carbonate, the retinyl acetate, the vitamin D3, the vitamin E, the ethoxyquin, and the carrier, in parts by weight, were fed and mixed in batches into a mixer proportionally in a layered feeding manner, while purified water equal to twice a total volume of additives was slowly added to obtain the first wet mix;
step 3, centrifugation: the premix obtained from step 2 was fed into a centrifuge with water and centrifuged for solid-liquid separation; solids were dried to obtain the first dry mix, and the first dry mix was loaded on a tray for later use;
step 4, a supernatant was mixed with the feed raw materials obtained from step 1 to obtain a second wet mix;
step 5, the second wet mix was heated and dried until the second wet mix became viscous to obtain the second semi-dry mix;
step 6, the viscous second semi-dry mix was extruded on a feed pellet pressing mill to obtain the second semi-dry mix pellets, and the second semi-dry mix pellets were loaded on a tray with the first dry mix; and
step 7, pellets formed by the adhesion of the second semi-dry mix pellets to the first dry mix were dried to obtain the finished pellet feed.

The feed raw materials in step 1 had a particle size smaller than 3 mm, each feed raw material was individually crushed, and all feed raw materials were mixed well in the mixer after crushed, such that the materials in each feed could be more uniform in the subsequent pelleting process.

The materials in step 2 had a particle size smaller than 1 mm, and all materials were mixed well in the mixer.

The centrifuge in step 3 was set at a rotational speed of 2,000-3,000 rpm.

The materials in step 4 had a particle size smaller than 1 mm, and the second wet mix needed to be wrapped with a cling film, reducing the water loss of the second wet mix when waiting for processing.

The drying in step 5 was set at 75-85° C., and the moisture of the second wet mix was controlled in a range of 15-20%.

The extrusion particle size of the feed pellet pressing mill in step 6 was 3-8 mm.

The drying in step 7 was divided into two stages of 200° C. and 80° C., and the materials were continuously turned over during the drying.

In the drying process, the outer layers of the first dry mix and the second semi-dry mix pellet were first dried at a temperature as high as 200° C. momently, so that the outer layer of the formed feed pellets could form a dense protective shell. Next, the inner layer of the feed pellets was further dried at a baking temperature of 80° C., so that the feed was relatively dry enough to be stored for a long time.

Although the embodiments of the present disclosure have been illustrated and described, it should be understood that those of ordinary skill in the art may make various changes, modifications, replacements and variations to the above embodiments without departing from the principle and spirit of the present disclosure, and the scope of the present disclosure is limited by the appended claims and their legal equivalents.

What is claimed is:

1. A pelleted complete mixed feed (PCMF) for improving lamb quality, wherein each kilogram of the PCMF comprises the following raw materials in parts by weight: 540-600 parts of corn, 85-115 parts of bran, 160-220 parts of stir-fried soybean meal, 50-75 parts of a safflower seed, 12-18 parts of a flaxseed, 80-90 parts of a sunflower seed hull, and 35-50 parts of a premix.

2. The PCMF for improving lamb quality according to claim 1, wherein each part by weight of the premix comprises the following components: 0.04-0.06% of copper sulfate pentahydrate, 0.18-0.23% of ferrous sulfate monohydrate, 0.24-0.32% of zinc sulfate monohydrate, 0.12-0.18% of manganese(II) sulfate monohydrate, 0.02-0.03% of calcium iodate monohydrate, 0.01-0.02% of sodium selenite, 0.01-0.02% of cobalt chloride monohydrate, 3-5% of magnesium oxide, 28-32% of calcium carbonate, 0.03-0.04% of retinyl acetate, 0.004-0.006% of vitamin D3, 0.50-1.0% of vitamin E, 0.25-0.35% of ethoxyquin, and 32-40% of a carrier.

3. A method for preparing the PCMF for improving lamb quality according to claim 1, comprising the following steps:
step S1, pretreatment of the feed raw materials: putting the corn, the stir-fried soybean meal, the bran, the safflower seed, the flaxseed and the sunflower seed hull in the parts by weight into a grinder for grinding;
step S2, pretreatment of the premix components: feeding and mixing the copper sulfate pentahydrate, the ferrous sulfate monohydrate, the zinc sulfate monohydrate, the manganese(II) sulfate monohydrate, the calcium iodate monohydrate, the sodium selenite, the cobalt chloride monohydrate, the magnesium oxide, the calcium carbonate, the retinyl acetate, the vitamin D3, the vitamin E, the ethoxyquin, and the carrier in parts by weight and in batches into a mixer proportionally in a layered feeding manner, while slowly adding purified water equal to twice a total volume of additives to obtain a first wet mix;
step S3, centrifugation: feeding the first wet mix into a centrifuge, centrifuging for solid-liquid separation, drying solids to obtain a first dry mix, and loading the first dry mix on a tray for later use;
step S4, mixing a supernatant with the feed raw materials obtained from step S1 to obtain a second wet mix;
step S5, heating and drying the second wet mix until the second wet mix becomes viscous to obtain a second semi-dry mix;
step S6, extruding the viscous second semi-dry mix on a feed pellet pressing mill to obtain a second semi-dry mix pellet, and loading the second semi-dry mix pellet on a tray with the first dry mix; and
step S7, adhering the first dry mix to a surface of the second semi-dry mix pellet, and conducting drying to obtain a finished pellet feed.

4. The method for preparing a PCMF for improving lamb quality according to claim 3, wherein the feed raw materials in step S1 have a particle size smaller than 3 mm, each feed raw material is individually crushed, and the feed raw materials are all mixed well in the mixer after crushing.

5. The method for preparing a PCMF for improving lamb quality according to claim 3, wherein the premix components in step S2 have a particle size smaller than 1 mm, and the components are all mixed well in the mixer.

6. The method for preparing a PCMF for improving lamb quality according to claim 3, wherein the centrifuge in step S3 is set at a rotational speed of 2,000-3,000 rpm, and centrifugation time is controlled in a range of 10-15 min.

7. The method for preparing a PCMF for improving lamb quality according to claim 3, wherein the second wet mix in step S4 is wrapped with a cling film.

8. The method for preparing a PCMF for improving lamb quality according to claim 3, wherein the drying in step S5 is set at 75-85° C., and a moisture of the second semi-dry mix is controlled in a range of 15-20%.

9. The method for preparing a PCMF for improving lamb quality according to claim 3, wherein a particle size for the feed pellet pressing mill in step S6 is set at 3-8 mm.

10. The method for preparing a PCMF for improving lamb quality according to claim 3, wherein the drying in step S7 is divided into two stages of 200° C. and 80° C., and materials need to be continuously turned over during the drying.

11. The method for preparing a PCMF for improving lamb quality according to claim 3, wherein each part by weight of the premix comprises the following components: 0.04-0.06% of copper sulfate pentahydrate, 0.18-0.23% of ferrous sulfate monohydrate, 0.24-0.32% of zinc sulfate monohydrate, 0.12-0.18% of manganese(II) sulfate monohydrate, 0.02-0.03% of calcium iodate monohydrate, 0.01-0.02% of sodium selenite, 0.01-0.02% of cobalt chloride monohydrate, 3-5% of magnesium oxide, 28-32% of calcium carbonate, 0.03-0.04% of retinyl acetate, 0.004-0.006% of vitamin D3, 0.50-1.0% of vitamin E, 0.25-0.35% of ethoxyquin, and 32-40% of a carrier.
